(19) United States
(12) Patent Application Publication
(10) Pub. No.: US 2014/0100593 A1
(43) Pub. Date: Apr. 10, 2014

(54) Plastic Surgical Instruments

(71) Applicant: Paul Sand, Redwood City, CA (US)

(72) Inventor: Paul Sand, Redwood City, CA (US)

(21) Appl. No.: 13/790,836

(22) Filed: Mar. 8, 2013

Related U.S. Application Data
(60) Provisional application No. 61/795,014, filed on Oct. 9, 2012.

(51) Int. Cl. A61B 17/32

(52) U.S. Cl. CPC A61B 17/32
USPC 606/167

(57) ABSTRACT

Disclosed herein are tools, systems, methods and surgical techniques for a disposable grasping, cutting, severing and/or biting surgical instrument having sharp cutting tips. Such instruments are lighter than their equivalent stainless steel instruments currently being used, and can be designed to employ a shearing/cutting or grasping mechanism that may be suited for specific cutting and sampling bone, cartilage and soft tissue. The surgical instrument includes a slideable upper body that may have an overmolded or removably connected cutting tip assembled onto an advancing face that translates along the handle body to produce a shearing, cutting or grasping action of tissue positioned within the jaws.

### RELATED APPLICATIONS

This application claims the benefit of U.S. Provisional Patent Application Ser. No. 61/795,014, entitled “Plastic Surgical Instruments,” filed Oct. 9, 2012, from which priority is claimed under 35 U.S.C. 119, and the disclosure of which is hereby incorporated herein by reference in its entirety.

### TECHNICAL FIELD

The invention relates to improved surgical tools, methods and systems for treating patients using inexpensive, easily manufactured and/or disposable/recyclable plastic surgical instruments.

### BACKGROUND OF THE INVENTION

Currently, surgical instruments used for cutting, severing or “biting” tissue and bone are made entirely from stainless steel. While the manufacture of such instruments can involve significant expense, these metal instruments can be re-sterilized numerous times and re-used for multiple surgeries involving different patients, which justifies their significant expense. In addition, the cutting edges of metal instruments can dull with use, depending on the type and extent of their use during surgery as well as the care with which the OR and sterilization department personnel use when handling and storing the instruments. When such metal instruments become sufficiently dull, they may be re-sharpened or, in the vast majority of cases, the instruments are discarded.

### SUMMARY OF THE INVENTION

There is a need for disposable grasping, cutting, severing and/or biting instruments for surgical procedures that remain sharp during each surgery. Desirably, such instruments would be lighter than their equivalent stainless steel instruments currently being used, and the component materials and processing requirements (i.e., manufacturing) would be less expensive than traditional stainless steel instruments. Such instruments would also desirably be disposable and/or recyclable, although the ability to re-sterilize the instruments would be desirable, should the need arise.

Various embodiments described herein include disposable, single use and/or re-sterilizable surgical instruments of various configurations molded or cast from a flowable material such as a plastic polymer or resin, with integral and/or replaceable metallic cutting and/or slicing features incorporated into the surgical instruments. In various alternative embodiments, features of the plastic instrument may be formed integrally with the metallic sub-components (i.e., overmolding, etc.), may be permanently bonded or otherwise irremovably secured to the metallic sub-components (i.e., locked or adhered), or the metallic subcomponents may be removable and/or replaceable for repair of the instrument and/or recycling of the component materials (i.e., plastic recycling and sharps disposal). Such surgical instruments may take the form of a wide variety of instruments, including designs similar to rongeurs, kerrison rongeurs, and/or kerrison punches.

In various embodiments, plastic surgical instruments may include a plurality of features that support a combination of grasping, severing, and cutting. The plastic instrument may include modular heads that may be desirably removably attached, or the instrument may be designed with bores or channels that allow insertion of cutting rods, instruments, surfaces, etc. In various embodiments, one or more of the cutting tips or other metallic portions could be modular and/or replaceable.

In an alternative embodiment, the plastic instrument may be designed with features that allow it to be used with applied energy systems such as electro-cautery and/or RF power sources for cutting, coagulating, desiccating, fulgurating or otherwise applying energy to tissue in a desired manner. It can be advantageous to design plastic instruments to accept and transmit electrical energies and/or currents. The plastic instrument may include one or more desired current pathways and could include replaceable and/or modular heads that may be connected to an electrosurgical generator to supply an electric current to the replaceable and/or modular heads. In other embodiments, the replaceable and/or modular heads may be changed or integrated with grasper features. Desirably, the plastic or non-metallic portions of the instrument will act as insulators for the surgeon during use.

The provision of cutting instruments comprised primarily of plastic or other nonmetallic and easily-moldable materials desirably fulfills the need for disposable grasping, cutting and/or biting instruments that are sharp for each surgery. Such disposable medical products offer many advantages for the clinicians, staff, and/or patients, which may be expressed with increased safety, convenience, or availability. Disposable instruments can be essential for streamlining patient care because the instrument can be readily available at a moment's notice, can be stored in a sterile form (and thus alleviate patients' and/or physicians' concerns with tool sterility), and such tools could save the clinician or staff valuable time, effort and expense by not requiring an autoclave and/or sufficient sterilization time to sterilize their equipment.

Disposable and/or recyclable instruments such as those described herein also offer medical device manufacturers various benefits over traditional stainless steel instruments. Such instruments may be produced at a fraction of the cost due to the way disposables are manufactured. Moreover, the plastic and metal components of such tools could be easily and conveniently recycled, and any small portions of the tools that cannot be recycled are easily compacted and/or crushed for disposal in landfills, burned in incinerators or disposed of using other traditional methods.

There are a wide variety of advantages that can be realized by the incorporation of plastic materials into surgical cutting instruments. For example, the use of flexible polymers in the design and manufacture of surgical instruments has the potential to significantly broaden the “design space” available and/or provide increased “design freedom” for the instrument designer as compare to traditional all-metal instruments. Specifically, plastics and polymers are particularly well-suited to the construction of flexible features and/or “living hinges” that are ill-suited to metals. Moreover, plastics are impact and dent resistant, and resist fracture or “shattering” of the component material during tool failure. Plastics are also more flexible than metals, which, allows them to be manufactured in tight tolerances and assembled together in “snap-fit” arrangements.

Other advantages in the use of plastics in surgical tools can include the fact that plastics and polymers are highly corrosion resistant, are impervious to many chemical compounds, and are typically electrically non-conductive. In addition, plastics are typically nonmagnetic, and can be safely utilized in the vicinity of strong magnetic fields, such as used in Magnetic Resonance Imaging equipment (MRI).

Plastics are typically radiolucent and do not scatter x-rays or other high-energy particles. However, where radiopacity is an important consideration, plastics may have controlled levels of radiopacity premixed or introduced into the polymer mixture to enhance fluoroscopic visualization.

As previously noted, plastic is lighter than metal, and plastics can be reinforced with core-throughs and kiss-offs for added strength. Plastics are versatile and can be used to create complex geometries, and many plastics are self-lubricating.

One extremely important consideration is that plastic is significantly cheaper than metal, and plastic parts can be manufactured for a fraction of the cost of their metallic counterparts. In addition, color can be integrated into the plastic material, and graphics and/or surface features can be integrated (i.e., molded) into the part, which prevents the graphic from ever coming off.

With the range of advantages that are experienced by clinicians, staff, patients and medical device manufacturers for the development of disposable cutting instruments, there exists a need to improve the cost, safety, convenience, and availability of plastic surgical instruments to satisfy these demands.

### BRIEF DESCRIPTION OF THE SEVERAL VIEWS OF THE DRAWINGS

FIG. 1 depicts a perspective view of one embodiment of a surgical instrument constructed in accordance with various teachings described herein;

FIG. 2 depicts an exploded perspective view of the surgical instrument of FIG. 1;

FIG. 3 depicts a partial perspective view of a tip of the surgical instrument of FIG. 1;

FIG. 4 depicts a partial perspective view of the tip of the surgical instrument of FIG. 3, from a different angle of view;

FIG. 5 depicts a partial perspective view of various surgical instrument tip designs;

FIG. 6 depicts a partial perspective view the surgical instrument of FIG. 4, with a portion of the tool depicted in shadow;

FIG. 6A depicts a perspective view of the upper slide cutting tip of FIG. 6 and associated handle body cutting tip;

FIGS. 6B through 6D depict views of various additional embodiments of handle body cutting tips;

FIG. 7 depicts a partial perspective view of the tip of the surgical instrument of FIG. 1, with a portion of the tool depicted in shadow;

FIG. 8 depicts a partial transparent view of a middle portion of the surgical instrument of FIG. 1, showing various moving parts;

FIGS. 9A through 9E depict various views of one alternative embodiment of an upper slide cutting tip support structure;

FIG. 9F depicts a perspective view of an additional alternative embodiment of an upper slide cutting tip support structure;

FIGS. 10A through 10C depict various views of one alternative embodiment of a handle body cutting tip and associated structure;

FIGS. 11A and 11B depict views of an upper slide cutting tip and associated handle body cutting tip in open and closing configurations;

FIG. 12A depicts a perspective view of one embodiment of a handle body cutting tip having an elongated support structure;

FIG. 12B depicts the handle body cutting tip of FIG. 12A embedded in an injection-molded surgical instrument body;

FIGS. 13A through 13E depicts various exemplary tip configurations for either or both of the cutting tips of the various embodiments described herein;

FIGS. 14A and 14B depict an exemplary operation of the surgical instrument of FIG. 1;

FIGS. 15A and 15B depict partial magnified views of the trigger body and upper slide FIG. 14A;

FIG. 16A depicts a side plan view of the trigger body of FIG. 14A;

FIG. 17A depicts a side plan view of one alternative embodiment of a trigger body with cam arrangement;

FIG. 17B depicts a side plan view of an upper slide body for use with the trigger body of FIG. 17A;

FIG. 17C depicts an assembled view of the trigger pivot head and slide body of FIGS. 17A and 17B;

FIG. 17D depicts a partial side plan view of an alternative embodiment of an upper slide body and handle body associated for use with the trigger body of FIG. 6C; and

FIGS. 18A through 21B depict views of various arrangements and designs for cutting tips.

### DETAILED DESCRIPTION OF THE INVENTION

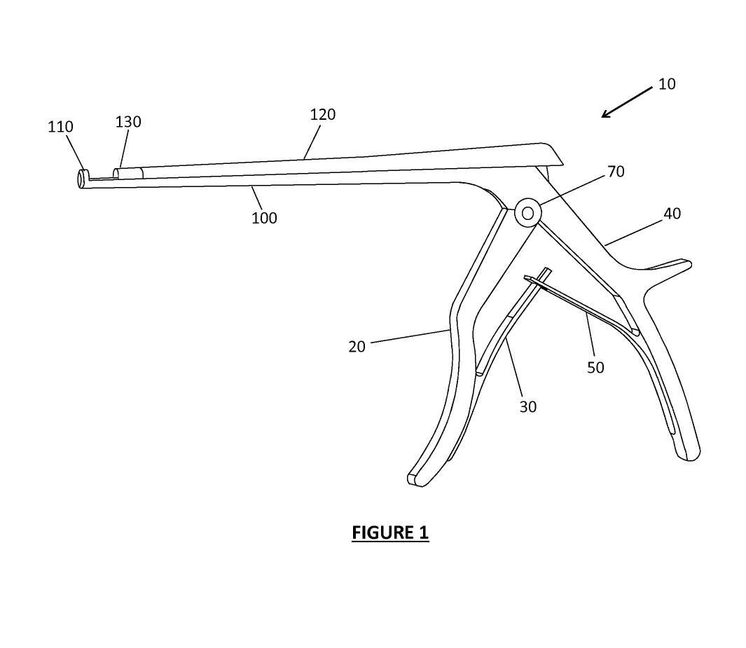

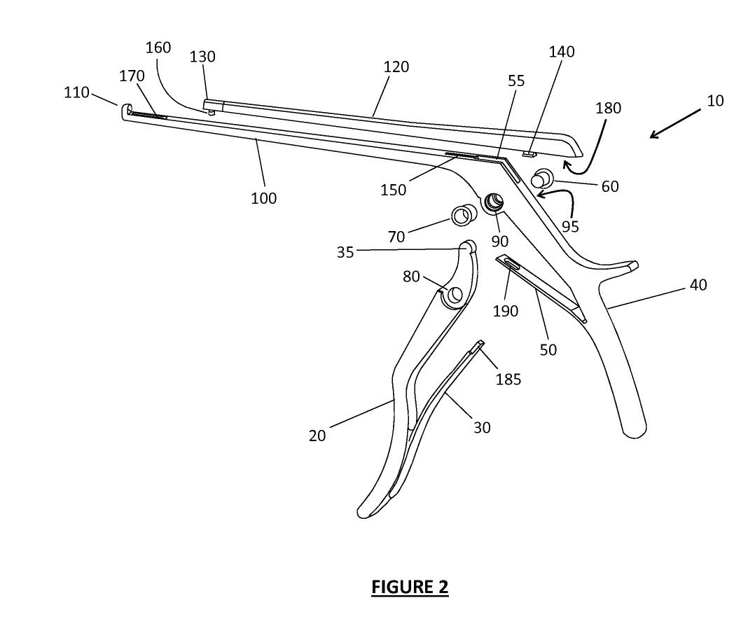

FIGS. 1 and 2 depict a perspective and an exploded perspective view of one embodiment of a surgical instrument 10 that incorporates various features of the present invention. The surgical instrument 10 includes a trigger body 20 and handle body 40. The trigger body 20 includes a trigger body spring 30 and a trigger pivot head 35. The handle body 40 includes a handle body spring 50, a central slot 55, a first handle body pivot hole 90 and a second handle body pivot hole 95.

A pivot screw body 60 and associated pivot nut 70 are provided that extend through a trigger pivot hole 80 and a handle body pivot hole 90, securing the trigger and handle together while allowing the trigger body 20 to rotate relative to the handle 40. The handle 40 further including a handle slide body 100 with a handle body cutting tip 110 (otherwise referred to as a footplate or anvil) formed at a distal end thereof. An upper slide body 120 of the surgical instrument 10 includes an upper slide actuator slot 180 (see FIG. 2) extending into the upper slide body 120, with an upper slide cutting tip 130 formed at a distal end of the upper slide body 120.

FIG. 2 highlights the upper slide body 120 which further includes an upper slide tee feature 140 which slides into a corresponding handle body tee feature 150. The upper slide body 120 also includes a distal tee pin 160 which slides into a corresponding handle body distal cutting tip slot 170.

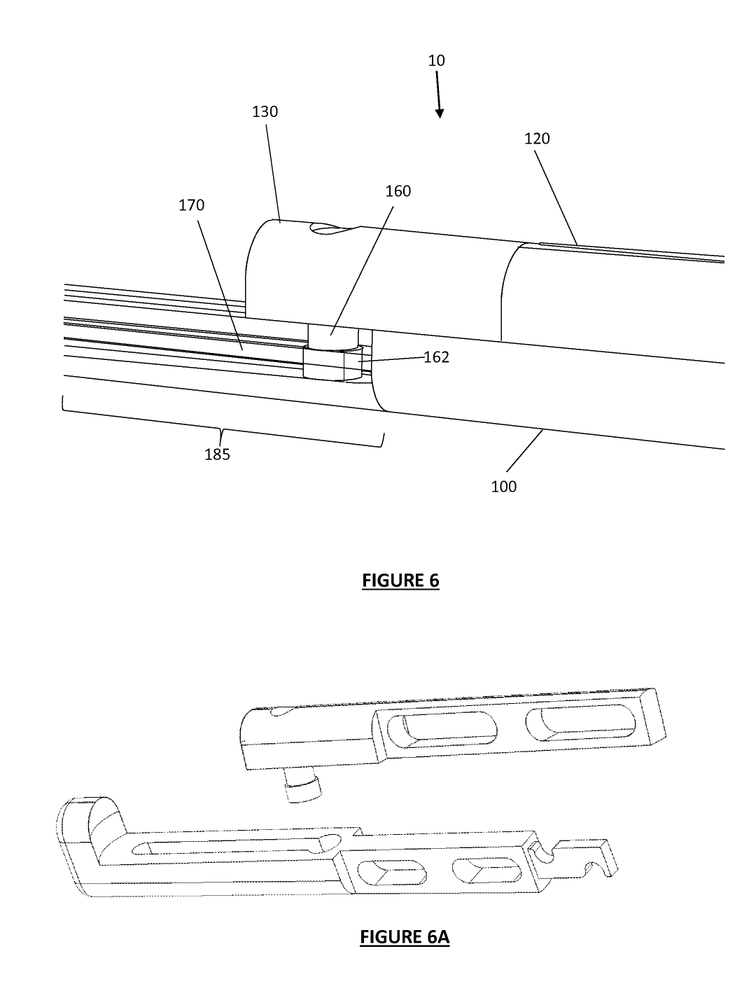

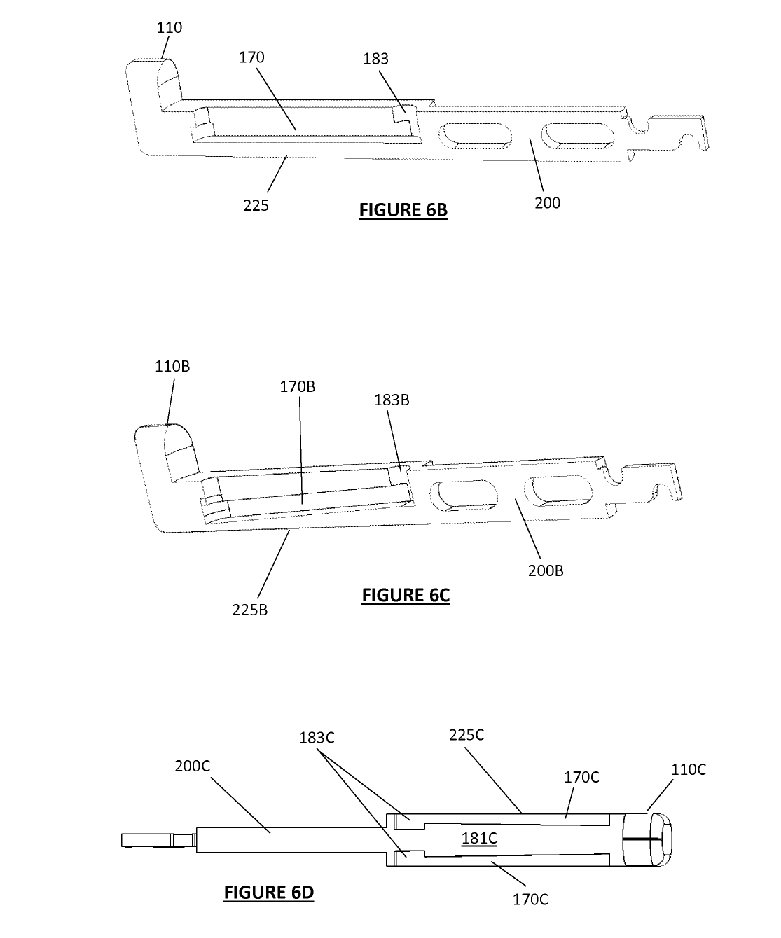

To assemble one exemplary embodiment of the surgical instrument 10, the trigger pivot head 35 can be slid through the central slot 55 of the handle body 40, with the pivot screw body 60 and pivot nut 70 extending into the first and second handle body pivot holes 90, 95 and through the trigger pivot hole 80. The upper slide body 120 is positioned adjacent the handle slide body 100, with the trigger pivot head 35 extending into the upper slide actuator slot 180 and the upper slide tee feature 140 sliding into the handle body tee feature 150 and the distal tee pin 160 and enlarged tee head 162 (see FIG. 6) sliding into the handle body distal cutting tip slot 170. The distal male end 185 of the trigger body spring 20 can then be inserted into a female slot 190 formed in the handle body spring 50.

Once the surgical instrument 10 is assembled, closing and/or squeezing of the trigger body 20 towards the handle body 40 will desirably induce the upper slide body 120 to slide along the handle slide body 100, with the upper slide cutting tip 130 approaching the handle body cutting tip 110. With sufficient compression, the cutting tips 130 and 110 will desirably meet, slide and/or “scissor” past each other (depending upon their relative size, shape and positioning), and thereby cut, sever and/or otherwise “bite” tissue and/or bone there between.

Once a desired cutting operation has been accomplished, releasing or lessening pressure on the trigger body 20 and handle body 40 will desirably permit the trigger body spring 20 and handle body spring 50 arrangement to flex and rotate the trigger body 20 relative to the handle body 40 away from the handle body 40, pulling and sliding the upper slide body 120 relative to the handle slide body 100 and displacing the upper slide cutting tip 130 away from the handle body cutting tip 110 in a known manner.

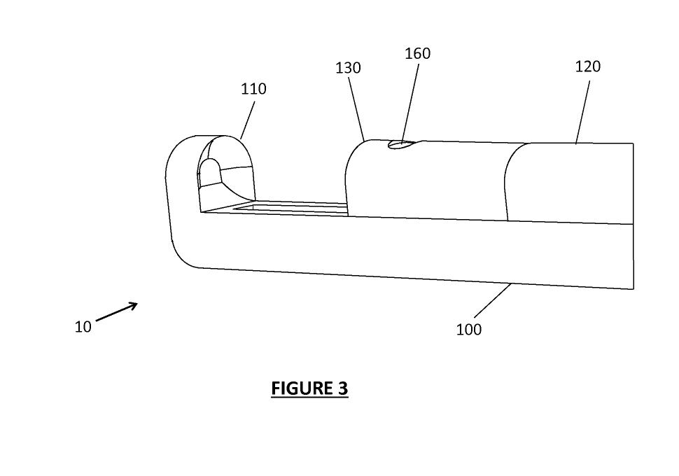

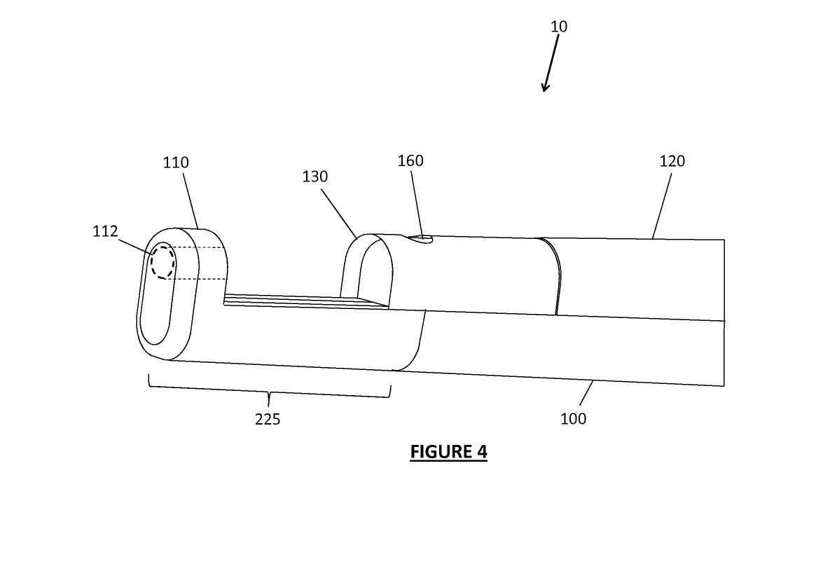

FIGS. 3 and 4 depict partial perspective views of a distal tip of the surgical instrument 10 from opposing angles, showing a handle body cutting tip 110, the upper slide cutting tip 130 and a distal tee pin 160. Desirably, as the handle (not shown) is actuated, the handle slide body 100 and upper slide body 120 will move relative to each other, bringing the handle body cutting tip 110 and upper slide cutting tip 130 towards each other and meeting in a known fashion to grasp, pinch, cut and/or sever a target material there between.

In one alternative embodiment, the handle body cutting tip 110 could optionally include a bore, channel or through-hole feature 112 where a wire, a guide wire, a hypotube, laparoscopic surgical graspers with loops or any standard diameter instrument that is commercially available in the operating room may be positioned or placed. The guide wire or grasper may be inserted from the back of the handle slide body 100 (not shown) and the upper slide body 120 (not shown) and align with the through-hole 112 should the surgeon require more precise placement of the tip of the surgical instrument embodiment or where manipulation of a body organ, tissue or bone prior to cutting is desired and/or required.

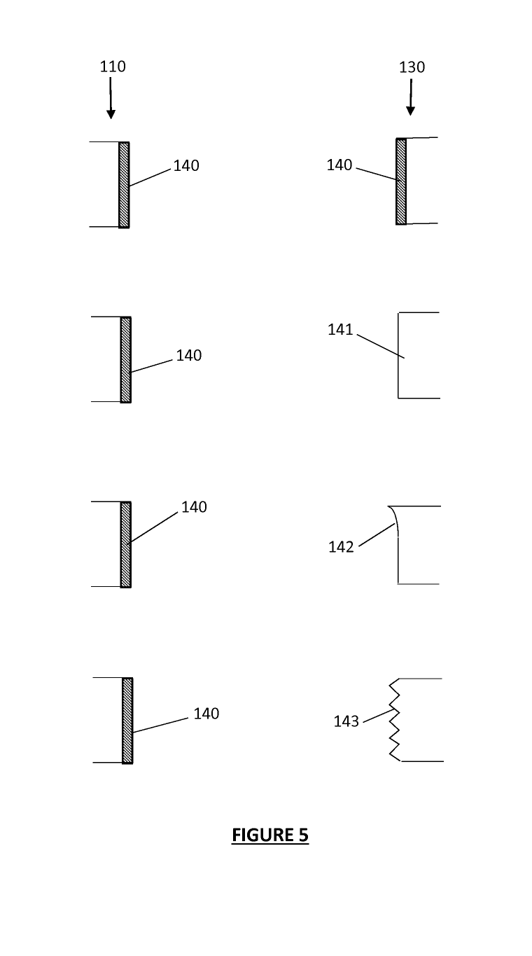

In various alternative embodiments, such as various exemplary embodiments shown in FIG. 5, the cutting tips 110 and 130 can comprise knife-edge (i.e., sharp) surfaces 140, or one or both surfaces can be relatively blunt or flat 141. Various other arrangements can include a tip surface having a projecting portion or “beak” 142 that can advantageously secure and/or pierce tissue intended to be cut. In other embodiments, one or more of the cutting tips can be serrated 143 or formed in a variety of shapes for differing surgical objectives. In one embodiment, it may be desirable to manufacture the cutting tips may 110 and 130 by metal injection molding. The tips may return to the manufacturer for further processing, such as sharpening the edges, shaping the edges, beveling the edges or add other features prior to assembling into the handle body cutting tip 110 and/or the upper slide cutting tip 130. Alternatively, the metal tips may be overmolded into the handle body cutting tip 110 and/or the upper slide cutting tip 130, and the manufacturer may decide to perform additional processing on the metal cutting tips prior to assembling the surgical instrument. In various alternative embodiments, one or more tips that are blunt, rounded, comprised of flexible materials and/or other arrangements can be provided, where desired

In various embodiments, the cutting surfaces may meet and contact at their respective sharp or blunt edges, or the surfaces may be sized and positioned such that one surface will ride over or past the other surface, inducing a sliding or scissoring effect by the surfaces that can be employed to create a desired cutting or severing action. In various alternative embodiments, the cutting surfaces may meet and contact at their respective sharp or blunt edges to grasp tissue, or any other target material intended there between. Alternatively, the edge may be curved in such a way that as the tips come together and contact at the top and as more pressure is applied, the handle slide body 100 (see FIG. 2 or FIG. 4) may be designed to flex downward, to have the cutting edges roll or slide across each other causing a slicing and or scissor action. Where the handle slide body 100 is formed of a sufficiently flexible material, such as a polymer, this flexibility can facilitate the flexing downward motion and subsequent rolling and/or sliding of the cutting edges relative to each other. In a desired embodiment, this can result in a deflection of the handle body cutting tip (or deflection of the handle body which reorients the cutting tip) such that the cutting tip 110 and the cutting tip 130 are no longer parallel, and thus the contact point propagates along the cutting tips, thereby creating a slicing or scissoring action.

FIG. 6 depicts a partial perspective view of the surgical instrument 10 and upper slide cutting tip 130, with a portion 185 of the handle slide body 100 in shadow. This view depicts how the distal tee pin 160 can slide within a track, groove or slot 170 formed within the handle slide body 100, which slidably secures the upper slide body 120 to the handle slide body 100 in a known fashion.

FIG. 6A depicts a perspective view of the upper slide cutting tip 130 of FIG. 6 and associated handle body cutting tip 110 prior to assembly.

FIG. 6B depicts a vertical cross-sectional view of the handle body cutting tip 110, L-shaped body 225 and handle body cutting tip support structure 200. This view highlights the slot 170 formed within the L-shaped body 225. The slot 170 includes a proximal opening 183 which is desirably sized to accommodate the enlarged head 162 of the distal tee pin 160 (see FIG. 6A). The slot 170 depicted in this embodiment may be designed to a specified length to allow for cutting, pinching, scissoring cut tips 110 and 130 to meet each opposing surface or to cross-over each opposing surface. However, other embodiments may vary the lengths and shapes the slot 170 for adjustability of the opposing cutting surfaces (i.e. they do not have to meet or cross-over) which could include the provision of opposing tip surfaces that, in a closed position, remain separated by a desired spacing. Such an arrangement could be particularly useful for use in handling and/or manipulating fragile tissue structure such as blood vessels, where excessive compression and/or crushing of the anatomy between the jaws of the instrument might be extremely undesirable.

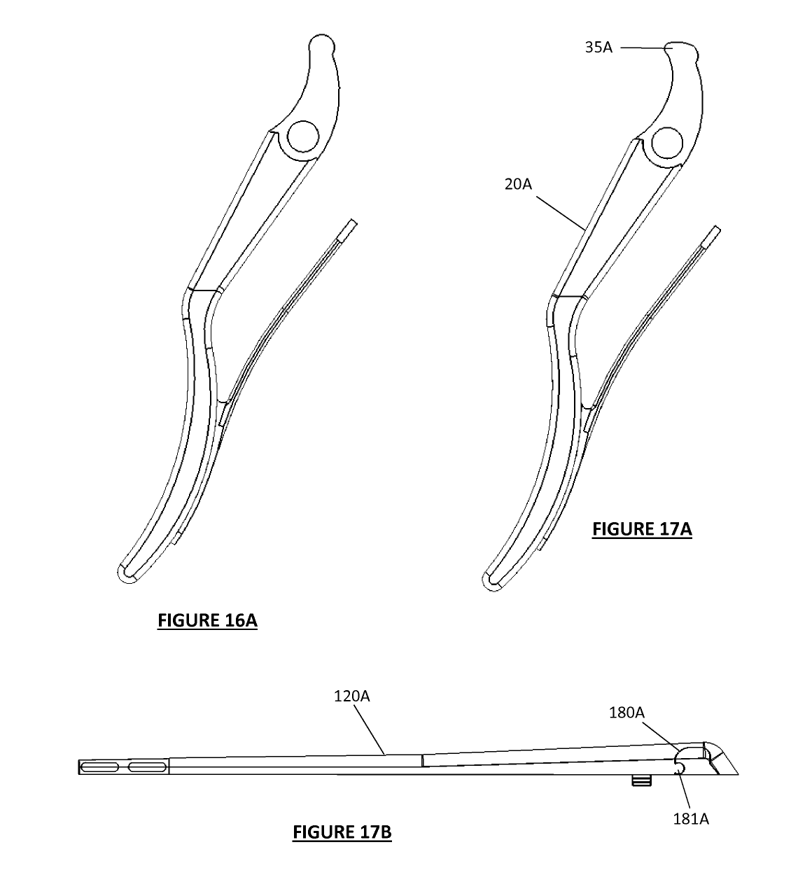

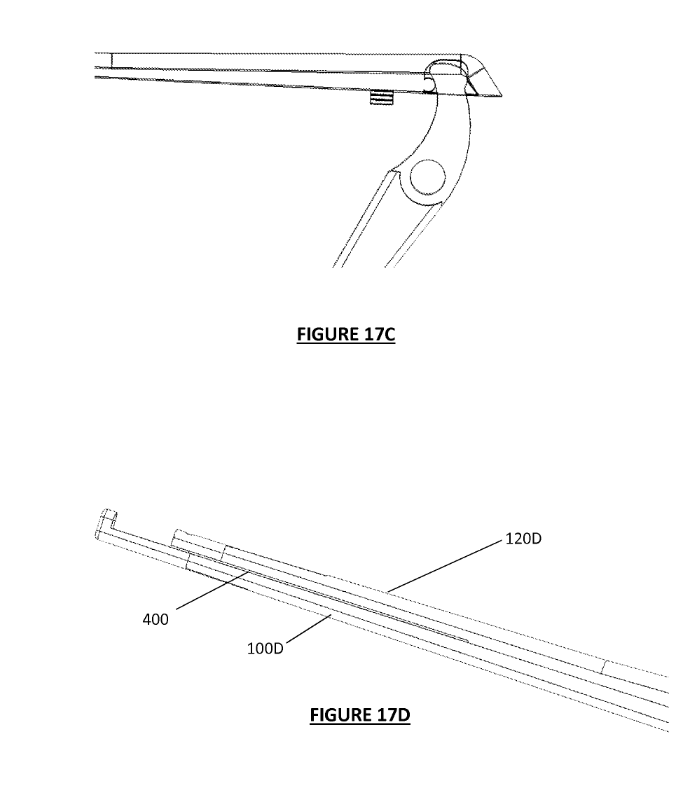

FIG. 6C depicts a vertical cross-sectional view of one alternative embodiment of a handle body cutting tip 1106, L-shaped body 225B and handle body cutting tip support structure 200B, showing the slot 170B formed within the L-shaped body 225B. The track 170B includes a proximal opening 183B which is desirably sized to accommodate the enlarged head 162 of the distal tee pin 160. In this embodiment, the slot includes a ramped section that progressively drops in the handle slide body 100B as it travels from the proximal opening 183B towards the cutting tip 1106, which desirably deflects the slide body cutting tip (not shown) in a desired manner towards the L-shaped body and, in various embodiments, causes the cutting tip to follow a non-linear, angled, curved, arctuate and/or otherwise complex path (depending upon the chosen ramp geometry) relative to the corresponding surface of the handle body cutting tip. In various embodiments, this may induce a downward slicing action which can significantly improve the ability of the tool to cut dense and/or fibrous tissues. The upper slide body 120D (see FIG. 17D) may also be designed to have a spaced region 400 between the slide body 120D and handle body 100D which allows a portion of the upper slide body 120D to flex as the slot pulls the cutting tip down.

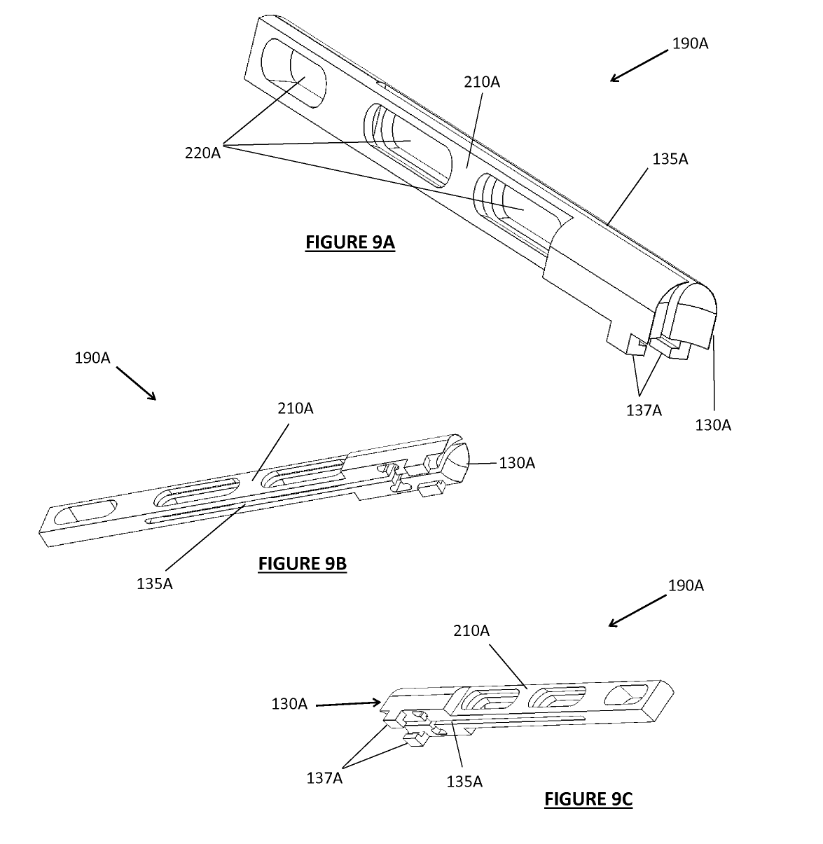

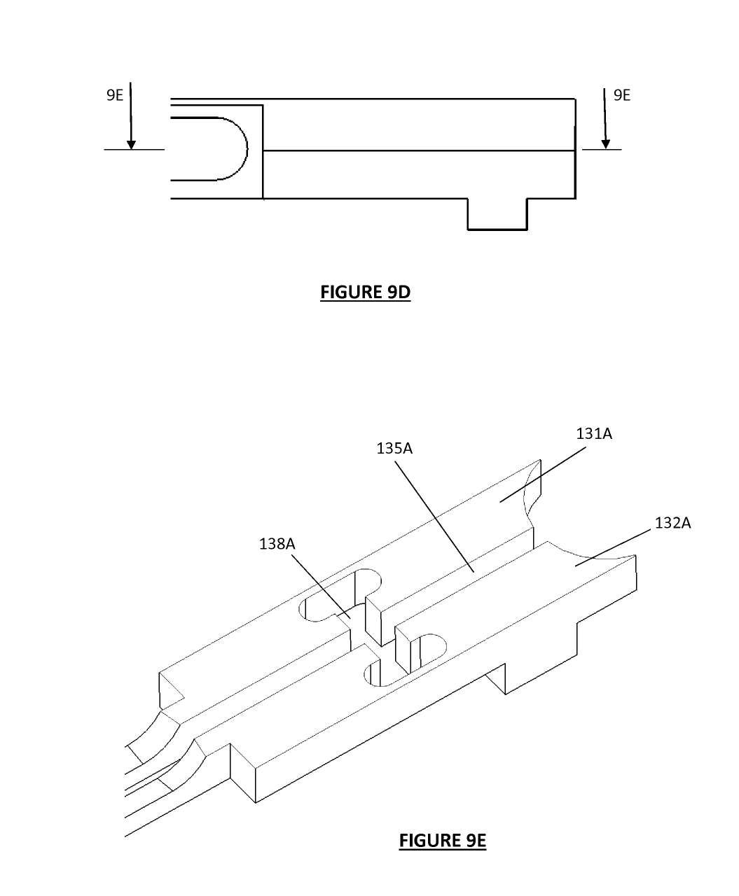

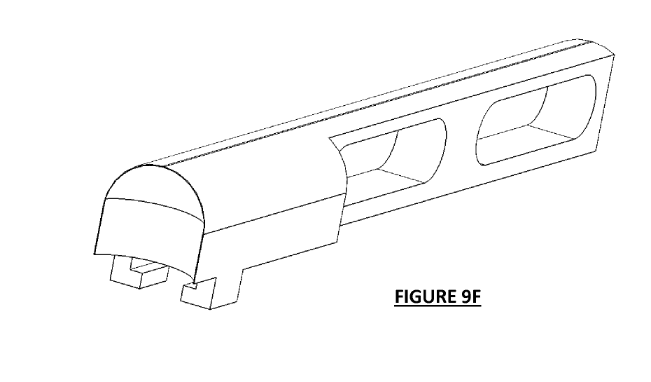

FIG. 6D depicts a top plan view of one another alternative embodiment of a handle body cutting tip 110C, L-shaped body 225C and handle body cutting tip support structure 200C, showing opposing slots 170C with a central ridge 181C that narrows in width as it extends from the proximal openings 183C to the cutting tip 110C. This embodiment may be particularly useful in conjunction with the upper slide cutting tip 130A of FIGS. 9A through 9E, in first and second tip portions 131A and 132A may flex relative to each other. In this embodiment, as the slide cutting tip travels towards the handle body cutting tip, the reduced width of the central ridge 181C can permit the first and second tip portions to flex towards each other, which may allow the edges of the cutting tips to assume a desired orientation relative to the handle body cutting tip 110C as well as potentially induce a “clipping” or scissoring action in addition to the slicing and/or compressing forces on the targeted anatomy.

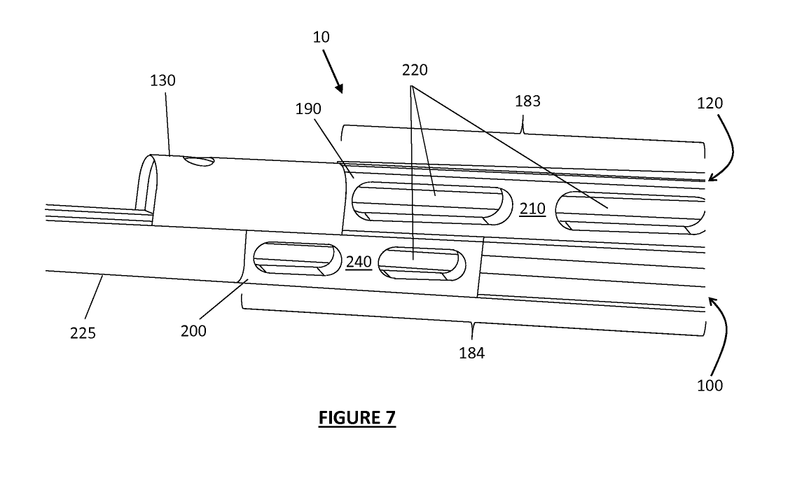

FIG. 7 depicts a partial perspective view of the surgical instrument 10 and upper slide cutting tip 130, with portions 183 and 184 of the upper slide body 120 and the handle slide body 100 in shadow. In this view, various features of the internal design of the upper slide cutting tip support structure 190 and the handle body cutting tip support structure 200 can be seen. The upper slide cutting tip support structure 190 includes an elongated central support body 210 connected to the upper slide cutting tip 130, with one or more openings or voids 220 formed in the support body 210. The voids 220 desirably reduce the amount of material to manufacture the support structure 190, increase the strength of the support structure, and also desirably facilitate securement of the support structure to the upper slide body 120. In one exemplary embodiment, the upper slide body 120 can be formed by overmolding a plastic material over the elongated central support body 210, with plastic material desirably extending through the various voids and interdigitating with the support body 210, thereby creating a unitary upper slide body 120 and upper slide cutting tip 130.

It should be understood that the overmolding material could include a variety of biomedical and/or biocompatible materials, including materials that exhibit superior properties for their intended use such as high performance polyethylenes, low friction polymers, flexible materials or hybrids of biomaterial combinations. In various embodiments, it would be desirable to employ the use of flowable materials known in the surgical arts, including plastics and polymers, latex, rubber, silicone, various ceramics and/or other known materials. In various embodiments described herein, the components of the instrument can primarily comprise a non-metallic material, with various features of either or both of the sliding and/or stationary components including some metallic, ceramic and/or other materials.

The handle slide body can be formed in a similar fashion, with the handle body cutting tip 110 including an L-shaped body 225 (see FIG. 4) that can be formed integrally with a handle body cutting tip support structure 200. The handle body cutting tip support structure 200 includes an elongated central support body 240 connected to the L-shaped body 225 of the handle body cutting tip, with one or more openings or voids 220 formed in the support body 240. The voids 220 desirably reduce the amount of material to manufacture the support structure 200, increase the strength of the support structure, and also desirably facilitate securement of the support structure to the handle body 100. In one exemplary embodiment, the handle body 100 can be formed by overmolding a plastic material over the elongated central support body 240, with plastic material desirably extending through the various voids and interdigitating with the support body 240, thereby creating a unitary handle slide body 100 and handle body cutting tip 110.

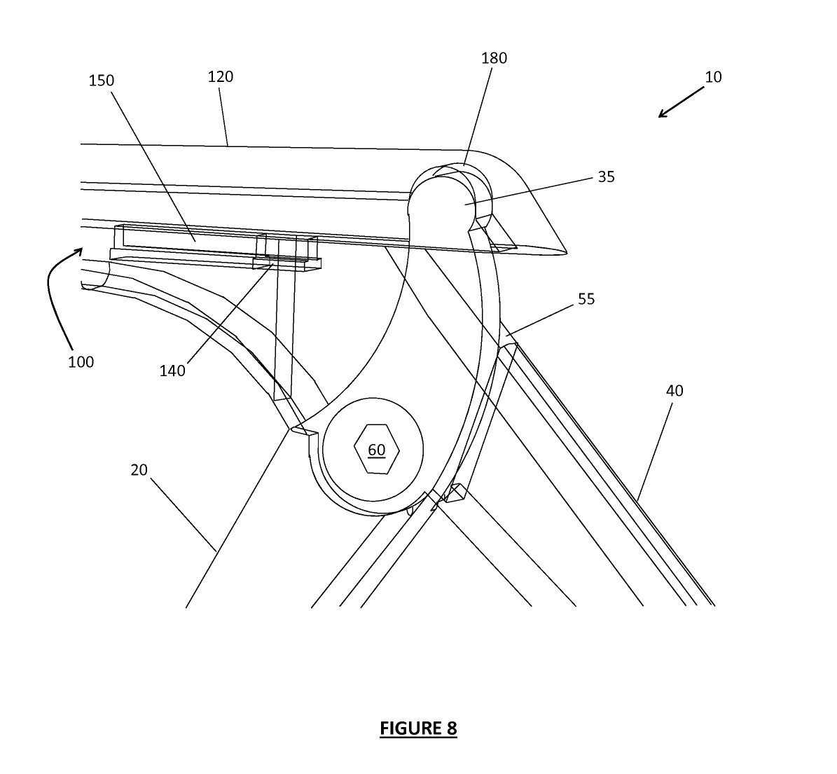

FIG. 8 depicts a partial perspective view of the surgical instrument 10, with various features of the handle body 40, the upper slide body 120 and the trigger body 20 shown in phantom. In this view, the surgical instrument 10 has been assembled, with the trigger pivot head 35 slid through the central slot 55 of the handle body 40, and the pivot screw body 60 and pivot nut (not shown) extending into and through the handle body pivot holes (not shown) and through the trigger pivot hole (not shown). The upper slide body 120 is positioned adjacent the handle slide body 100, with the trigger pivot head 35 extending into the upper slide actuator slot 180 and the upper slide tee feature 140 sliding into the handle body tee feature 150.

Once assembled in this manner, closing and/or squeezing of the trigger body 20 towards the handle body 40 will desirably induce the upper slide body 120 to slide along the handle slide body 100, with the upper slide cutting tip and handle body cutting tip (not shown) approaching and contacting or sliding past one another. As previously noted, with sufficient compression, the cutting tips will desirably meet and/or “scissor” past each other (depending upon their relative size, shape and positioning), and thereby cut, sever and/or otherwise “bite” tissue and/or bone between there between.

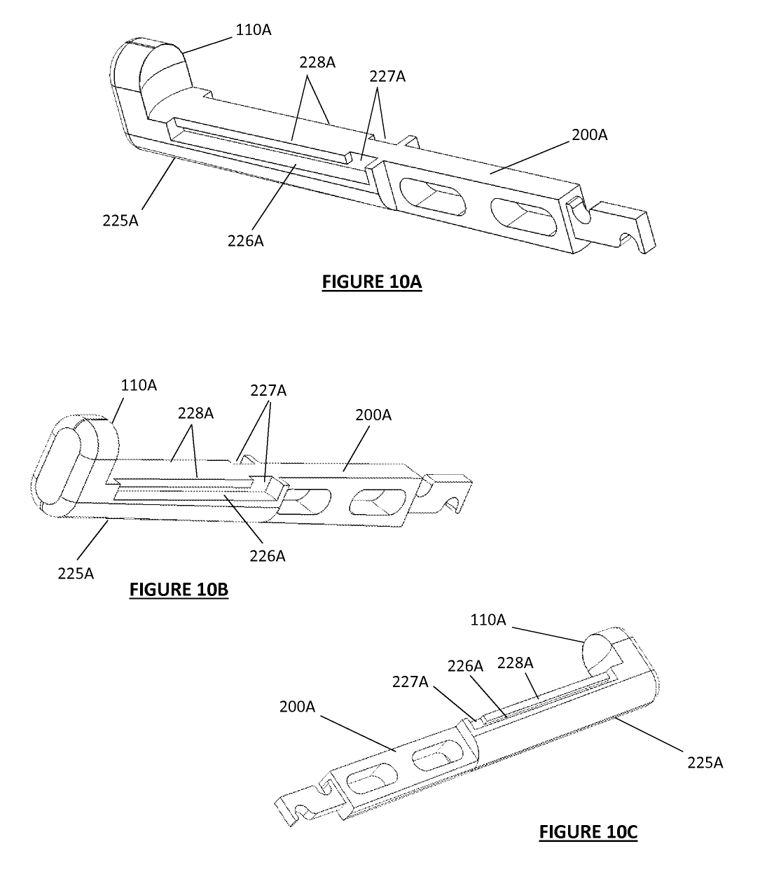

FIGS. 9A through 9C depict various views of one alternative embodiment of an upper slide cutting tip support structure 190A. This support structure 190A includes an elongated central support body 210A connected to the upper slide cutting tip 130A, with one or more openings or voids 220A formed in the support body 210A. The voids 220A desirably reduce the amount of material to manufacture the support structure 190A, increase the strength of the support structure, and also desirably facilitate securement of the support structure to the upper slide body 120A. A central slot 135A can be seen extending through the upper slide cutting tip 130A and support body 210A, which desirably reduces the amount of metal incorporated into the upper slide as well as facilitates additional interdigitation and flow of the overmolded plastic around the support structure 190A. In addition, the cutting tip 130A includes a pair of guiding tabs 137A that slide into a pair of slots 226A (see FIG. 10A) In one exemplary embodiment, the upper slide body 120A can be formed by overmolding a plastic material over the elongated central support body 210A, with plastic material desirably extending through the various voids and interdigitating with the support body 210A, thereby creating a unitary upper slide body and upper slide cutting tip.

In various alternative embodiments, reinforcing materials or “strips” could be included to stiffen and/or strengthen the various tool components described herein. For example, the handle body could include one or more longitudinally extending strips or fibers that bind with and extend through the flowable material, in a manner similar to reinforcement using cement re-bar or composite materials. Other embodiments could include reinforcing metallic strips or features, such as strips of metal over various portions of the handle (i.e., a strip of metal over the bottom heel of the handle and extending to and past the pivot location) which can be positioned outside the flowable material, positioned adjacent to the flowable material (i.e., on the surface) or that can be overmolded during the injection molding process.

FIG. 9D shows a partial side plan view of the cutting tip 130A and support structure 190A, with FIG. 9E depicting a cross-sectional view of the tip 130A taken along plane 9E-9E of FIG. 9D. In the exemplary embodiment, the cutting tip 130A includes the central slot 135A and also includes a centrally-positioned dumbbell-shaped void 138A. In one embodiment, the dumbbell shaped slot 138A (see FIG. 9E) may be designed to accept and capture any “I” beam shaped part (not shown) that can be sized to keep or maintain the guiding tabs 137A (see FIG. 9C) on the lower track, desirably prevent movement, and/or may allow the cutting tip 130A to translate toward each other until it contacts the handle body cutting tip 110A (see FIG. 10A) to achieve a scissors action.

FIG. 9F depicts another alternative embodiment of a cutting tip 130B and support structure 190B formed entirely of a metallic, ceramic or sufficiently hard plastic material, which does not include an internal slot and void arrangement of FIGS. 9A through 9C.

FIGS. 10A through 10C depict various views of one alternative embodiment of a handle body cutting tip 110A, including an L-shaped body 225A that is formed integrally with a handle body cutting tip support structure 200A. The L-Shaped body includes a pair of slots 226A, with each slot including a tab opening 227A and an elongated retention tab 228A that desirably accommodates and retains the corresponding guiding tabs 137A of the upper slide cutting tip 130A.

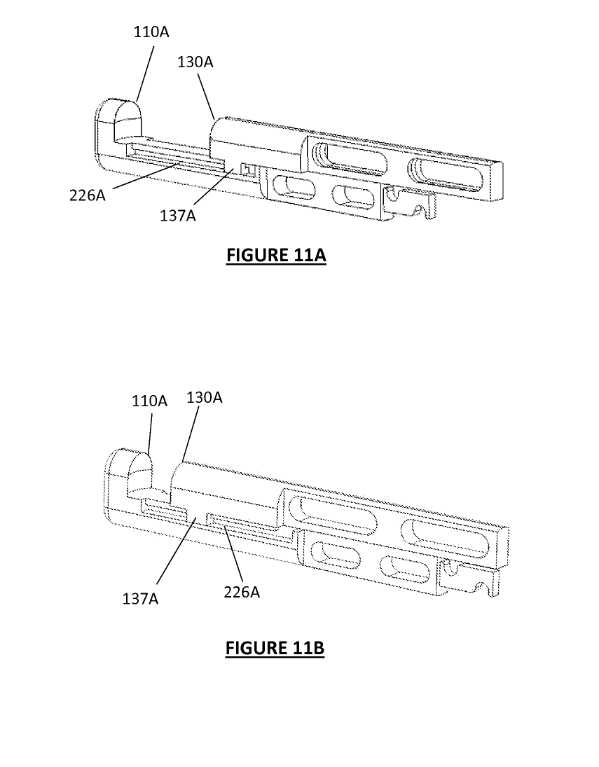

FIGS. 11A and 11B are views depicting the closing motion of the upper slide cutting tip 130A and handle body cutting tip 110A in an open configuration (FIG. 11A) and in a closing configuration (FIG. 11B). Desirably, the guiding tabs 137A are retained within the slots 226A and guide the motion of the slide cutting tip 130A as it moves towards the body cutting tip 110A. This arrangement desirably ensures good alignment of the cutting edges even when cutting hard or dense tissue (i.e., bone) as well as softer or fibrous tissues.

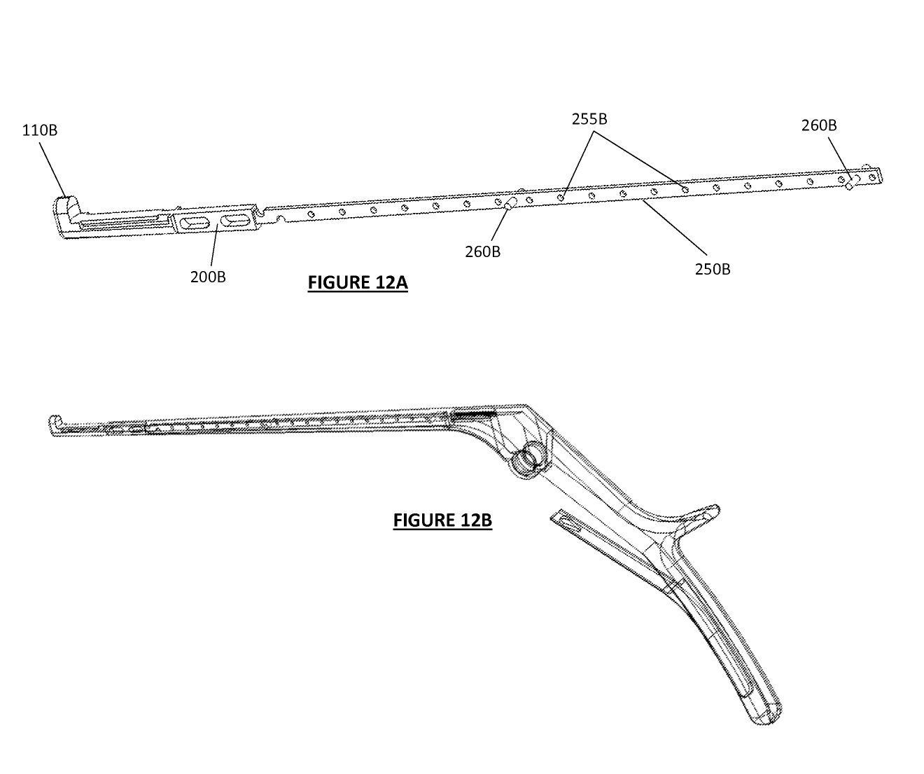

FIG. 12A depicts a perspective view of an alternative embodiment of a handle body cutting tip 1108, which includes an elongated support member 250B extending from the handle body cutting tip support structure 200B. Desirably, the elongated support member 250B provides additional stiffening and support to the handle body slide, and is desirably encased within the flowable plastic material during the injection molding process that can be employed to create the handle body. To facilitate interdigitation of the flowable material, the elongated support member 250B may include opening or voids 255B along its length, which can desirably reduce the amount of material used to create the member 250B, potentially stiffen the member 250B and/or the handle slide body (in a manner similar to concrete rebar), and allow for “flow-through” of the flowable material during the manufacturing process. To stabilize the support member 250B within the mold during plastic injection, one or more horizontal tabs 260B can be provided (with corresponding vertical tabs included alternatively or in addition, if desired). FIG. 12B depicts one view of a handle body including the embodiment of FIG. 12A embedded at least partially therein.

In one alternative embodiment, the elongated support member 250B could alternatively comprise a highly flexible metal strip or “string” with enlarged portions (i.e., beads) positioned along its length (not shown). A mold gate for injecting a flowable plastic material to create the handle body could be positioned proximate the cutting tip, allowing the flowable material entering the mold to flow along the string of beads and straighten the string as the molten plastic is injected into the mold. In another alternative embodiment, the elongated support member could comprise a metal track or guide along which the slide member (not shown) can travel during cutting and retraction actions, with the track at least partially embedded in the polymer (with track portions at least partially exposed) as previously described.

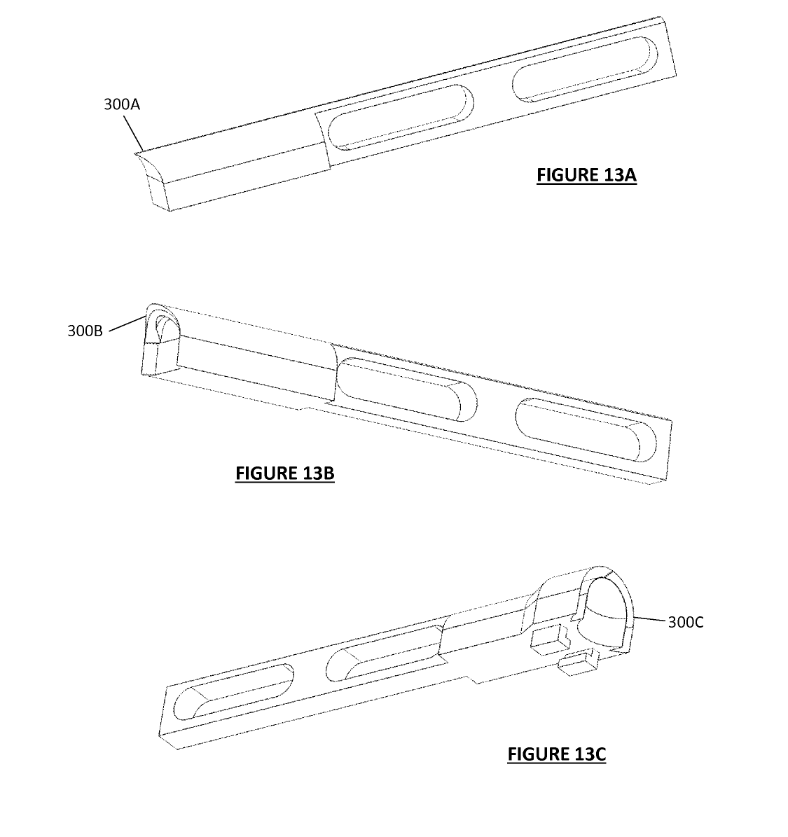

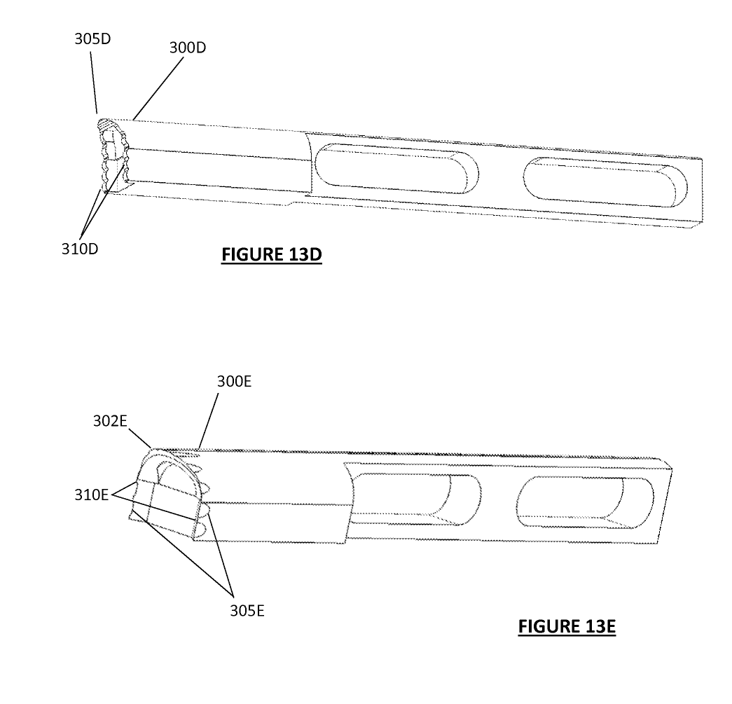

FIGS. 13A through 13E depicts various exemplary tip configurations that can be provided for one or more of the cutting tip described herein. FIG. 13A depicts a punch tip 300A which can be used to perforate tissue. Alternatively, the punch tip 300A can pierce and secure tissue for a variety of reasons, including for holding and manipulating the patient's anatomy, securing tissue for other operations (i.e., cutting with the same or another surgical tool) and/or to create a pathway or opening through a variety of tissue structures. In addition, the punch tip 300A significantly reduces the cross-sectional area of the tip, which commensurately increases the contact and penetrating pressure that can be generated on tissue by the tool.

FIG. 13B depicts a modified punch tip or “hawks bill” tip 300B, which is designed to pierce and cut tissue along an accurate plane. By angling the cutting surface, the hawk's bill tip 300B provides for a significant sliding or slicing cutting action near the tip, which significantly increases the ability of the tool to cut harder and fibrous tissue.

FIG. 13C depicts a dual-action cutting tip 300C having a pair of cutting edges. In various embodiments, the dual edges of the cutting tip can interact with corresponding dual cutting edges on the handle body cutting tip (not shown), or alternatively a flat or recessed handle body cutting tip. In one alternative embodiment, the handle body cutting tip can include a single edge that fits between the dual edges of the dual-action cutting tip 300C, which significantly increases the cutting ability of the various cutting tip edges by combining a scissoring action with the cutting action of the sharp edge(s). This design could also be useful for trapping, compressing, smashing and/or cutting anatomical features for a variety of surgical reasons.

FIG. 13D depicts a modified punch or corrugated tip 300D having a piercing portion 305D and serrated or wavered edges 310D. This tip 300D can be particularly advantageous in obtaining biopsies or for cutting fibrous tissue requiring significant edge strength as compared to a standard knife edge tip.

FIG. 13E depicts a hawk's bill-type tip 300E that includes a piercing tip 302E, scalloped or thinned-edge sections 304E and thicker edge-sections 310E. The scalloped sections desirably reduce the profile of the cutting blade (and potentially decrease the profile and “sharpness” of the blade section) while the thicker edge sections strengthen and add stiffness to the cutting edge. This arrangement may be particularly useful for cutting of extremely hard tissues like cortical bone or other structures. In addition, the piercing tip 302E can be especially useful for cutting of tissues having difficult, hard to reach profiles or that may be especially “slippery” for the tool to grasp.

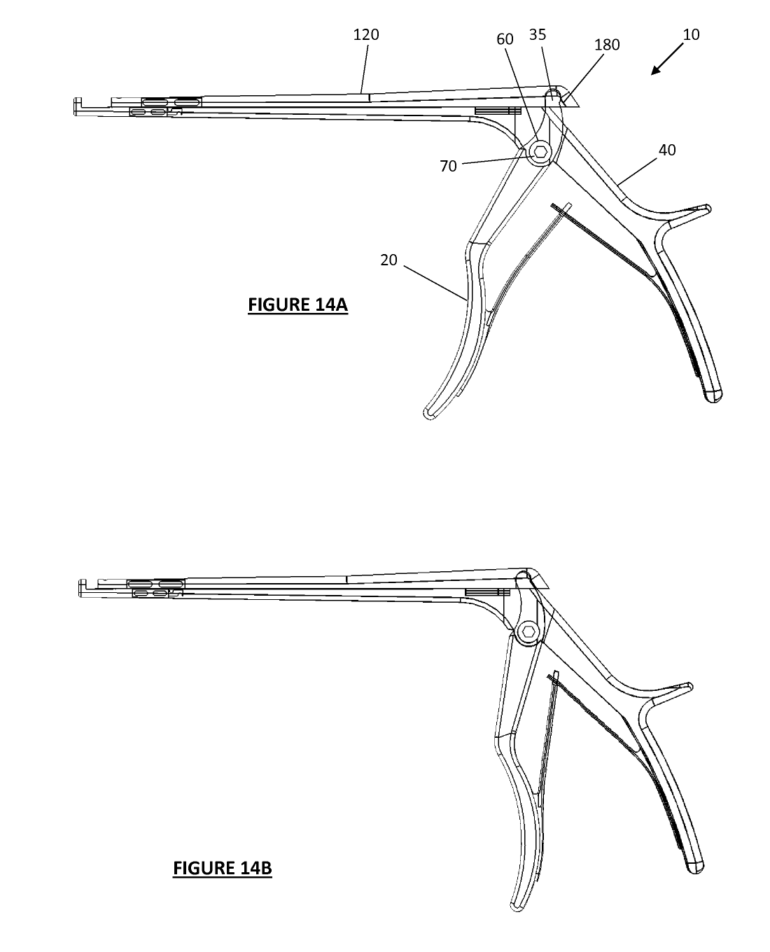

FIGS. 14A and 14B depict an exemplary operation of the surgical instrument 10, with the trigger body 20 being compressed relative to the handle body 40, which induces the trigger body 20 to rotate relative to the handle body 40 around the pivot screw body 60 and associated pivot nut 70, which pushes the trigger pivot head 35 (which extends through the central slot 55 of the handle body 40) into contact with an anterior wall of the upper slide actuator slot 180 and forcing the upper slide body 120 forward relative to the handle body 40 (see FIG. 14B). Continued compression of the trigger body 20 will desirably bring the cutting tips into contact or close proximity with each other to cut, sever or otherwise contact the targeted tissues or bone in a desirable manner.

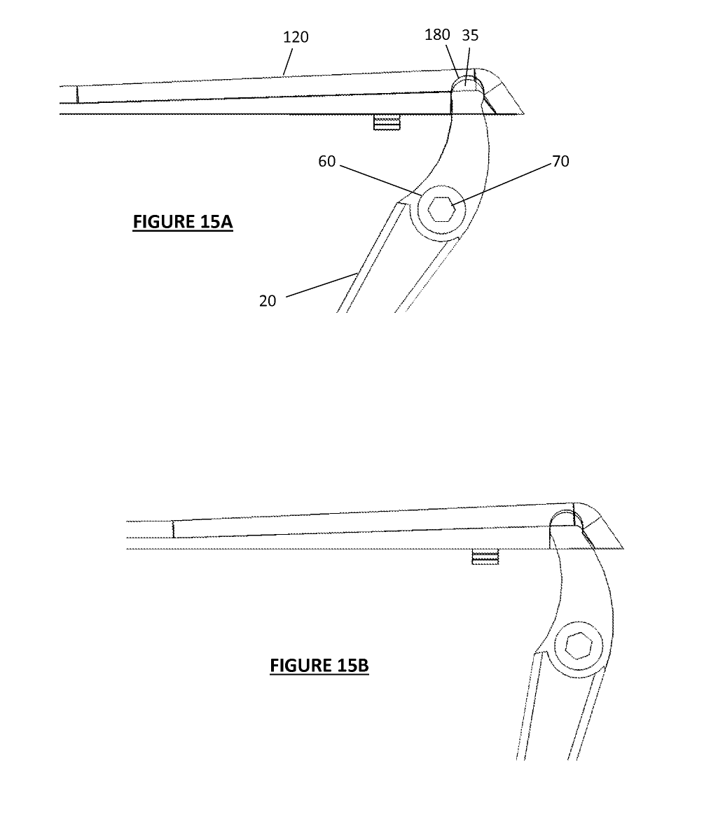

FIGS. 15A and 15B depict partial magnified views of the trigger body 20 and upper slide body 120 of FIGS. 14A and 14B, respectively. In an exemplary embodiment, the trigger pivot head 35 may designed to closely match the dimensions of the upper slide actuator slot 180 to prevent loosening during actuation, ease of assembly, and removal. In other embodiments, the trigger pivot head may have features that assist with connection to the upper slide actuator slot 180, such as a hook (see FIG. 17B), to prevent unintended disassembly. FIG. 16A depicts a side plan view of the trigger body 20 of FIGS. 14A and 14B.

FIG. 17A depicts a side plan view of one alternative embodiment of a trigger body 20A which includes a cam arrangement that desirably causes the upper slide to move non-linearly during compression of the trigger body. FIG. 17B depicts a side plan view of an upper slide body 120A for use with the modified trigger body 20A of FIG. 17A. In this embodiment, the trigger body 20A includes a modified trigger pivot head 35A which interacts with a cam 181A positioned within the upper actuator slot 180A of the upper slide 120A. Depending upon the rotational position of the trigger pivot head 35A and the cam 181A, movement of the trigger body 20A can cause greater or lesser resulting movement of the upper slide body 120A relative to the handle (not shown). FIG. 17C depicts a partial view of the trigger pivot head 35A and upper slide body 120A.

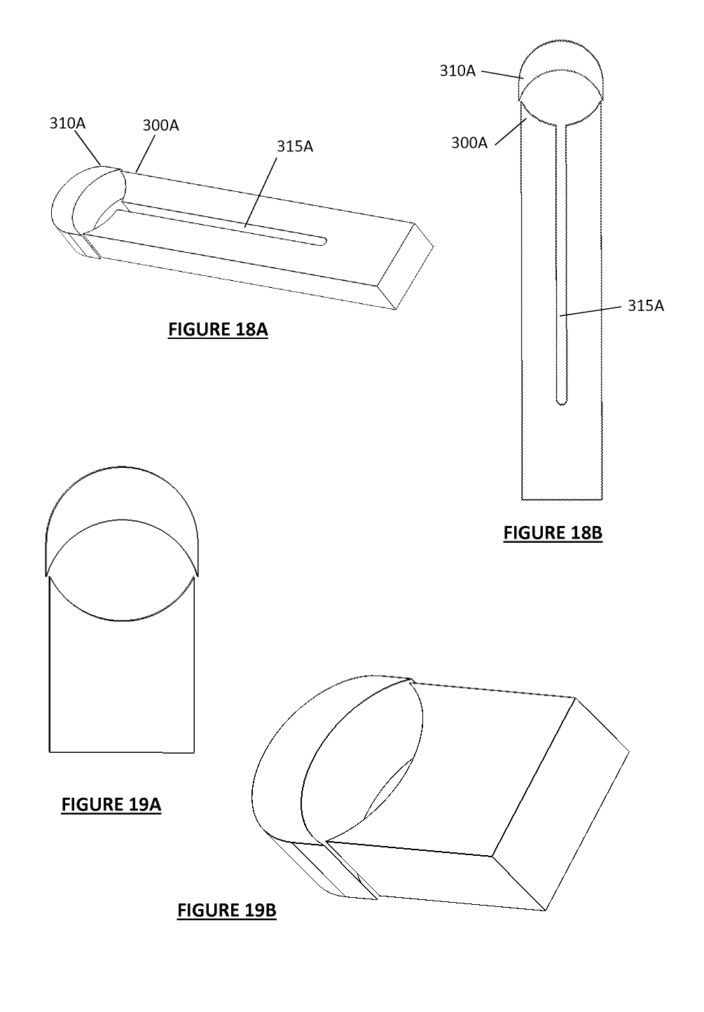

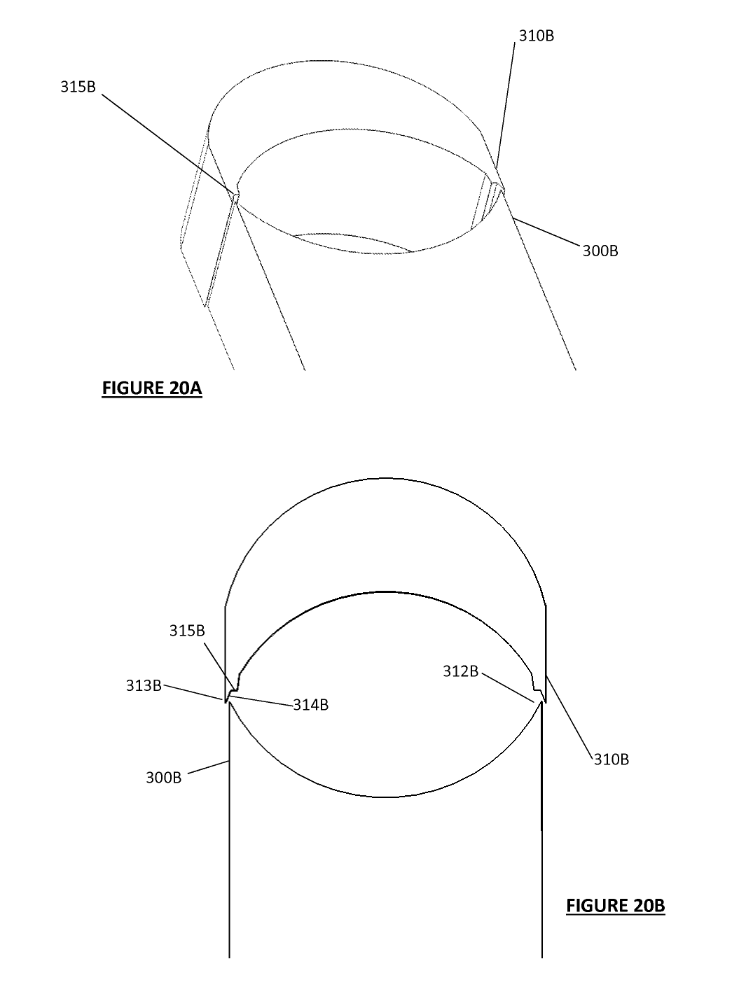

FIGS. 18A through 21B depict views of various cutting tip arrangements and designs suitable for incorporation into the various embodiments described herein. For example, the embodiment of FIGS. 18A and 18B include corresponding edged cutting surfaces where the edges of the upper slide cutting tip 300A are overlapped by the cutting surfaces at the edges of the handle body distal cutting tip 310A. This embodiment also includes a central slot 315A which desirably allows flexing of the sections of the upper slide cutting tip 300A when they come into contact with the handle body distal tip 310A. FIGS. 19A and 19B depict an embodiment of cutting tips similar to that of FIGS. 18A and 18B, but without a central slot.

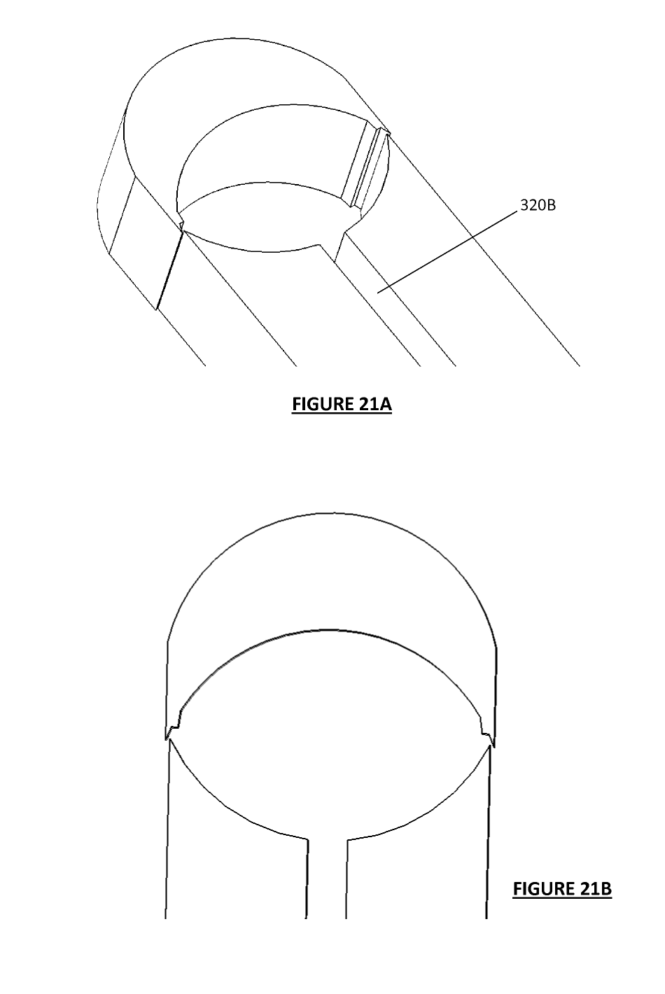

FIGS. 20A through 20B depict one embodiment of a cutting tip section having a modified flat or “anvil” type cutting arrangement. In this embodiment, the edges of the upper slide cutting tip 300B are overlapped by the cutting surfaces of the handle body distal cutting tip 310B. A flattened tip section or anvil 315B (or 315C) is also provided on the handle body distal cutting tip 310B (or 310C), which desirably interacts with the upper slide cutting tip 300B (or 300C) when the upper slide cutting tip 300B (or 300C) is sufficiently advanced. In use, a sharp edge 312B of the upper slide cutting tip 300B will desirably approach a corresponding edge 313B of the handle body distal cutting tip 310B, with targeted anatomy desirably positioned there between. As the sharp edge 312B is further advanced, the anatomy will desirably be wedged between the sharp edge 312B and the angled cutting surface 314B. Further advancement of the sharp edge 312B will desirably advance the edge to the flattened section or anvil 315B. This action desirably provides a composite cutting action to the anatomy, which can comprise combinations of nipping, cutting, punching and slicing of the anatomical structures between the cutting tips. FIGS. 21A and 21B depict an embodiment of cutting tips similar to that of FIGS. 20A and 20B, but with a central slot 320B.

If desired, various embodiments herein could include sensors or other features integrated into the various tool portions and/or overmolded therein. In various alternative embodiments, one or more wires or power supplies could be embedded or overmolded by flowable material, connected or otherwise linked to the metallic portions of the device (i.e., the cutting tips) and monopolar and/or bipolar energy provided there through to enable cauterization or other energy application to anatomical tissues by one or more of the metallic subcomponents. Because the operator's hand would be protected from such energy by the nonconductive nature of the flowable material (assuming the incorporation of such insulating material), no additional shielding would be required, unlike currently-available cautery instruments. In one embodiment, it may be advantageous to incorporate a through-hole or a bore in the upper slide body 130 and/or the handle slide body 100, where the bore may be lined and/or filled with a material that allows conduction of electricity to the cutting tips (not shown). The back of the surgical instrument may be designed to accommodate a fixed or removable coupling that can be attached to the electrocautery machine (not shown). An independent electrical conductor may extend from the electrocautery machine to be removably connected to the surgical instrument coupling to potentially transmit electrical energy through the through-hole or bore to the cutting tips to cut tissue. Alternatively, the through-hole or the bore may be lined with some insulation tube to separate the surgical instrument from the conduction of electrical energy to prevent the electrical conduction to pass along the surgical instrument, the surgeon or to the patient. The electrical insulation tubing may comprise of any of the suitable low dielectric materials, such as PTFE, TFE, polyimide, silicone, and/or polypropylene.

In other alternative embodiments, the instrument may include other actuating mechanisms to achieve linear motion. For example, the instrument may incorporate various other mechanical lead screw systems, cylinders with pistons powered by compressed air, hydraulic cylinders with pistons to provide large forces and quick strokes for hard tissue, cartilage or bone. Other embodiments may include other types of rotary motion to achieve linear motion, such as cam or rack and pinion designs. Also, various lead screws, roller screws, and ball bearing sliders may also be desirable.

In at least one exemplary embodiment, a surgical instrument kit can be provided that includes a surgical instrument having a plurality of upper slide tips, with the upper slide tips including multiple copies of a given cutting tip design. Alternatively, a surgical instrument kit could include a surgical instrument having a plurality of upper slide tips, with the upper slide tips including differently shaped and/or sized cutting tips. The various slide tips of such kits could be replaced when the cutting tip became damaged and/or dulled, or if a different cutting and/or manipulating action were desired by the physician. If desired, the handle and/or actuating lever/mechanism of such surgical instruments could be formed of a durable material such as metal, while the upper slide could comprise a hybrid of a plastic body integrally formed with metallic cutting tips, such as those described herein.

### INCORPORATION BY REFERENCE

The entire disclosure of any publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Various modifications to the embodiments described will be readily apparent to those skilled in the art, and the generic principles defined herein can be applied to other embodiments and applications without departing from the spirit and scope of the present invention as defined by the appended claims. The true scope of the invention is thus indicated by the descriptions contained herein, as well as all changes that come within the meaning and ranges of equivalency thereof, and the present invention is not intended to be limited to the embodiments shown, but is to be accorded the widest scope consistent with the principles and features disclose herein.

What is claimed is:

1. A surgical rongeur comprising:
a stationary handle connected to a stationary arm;
a moveable arm slidingly connected to said stationary arm;
a moveable handle rotatably coupled to said stationary handle, with a distal portion of said moveable handle coupled to said moveable arm such that said moveable arm slides relative to said stationary arm in response to rotation of said moveable handle relative to said stationary handle;
the moveable arm having a cutting tip;
at least a portion of the moveable arm comprising a non-metallic material; and
at least a portion of the cutting tip comprising a metallic material having a cutting edge.

2. The rongeur of claim 1, further comprising a cutting surface formed on a portion of the stationary arm, the stationary arm comprising a non-metallic material and the cutting surface comprising a metallic material, wherein rotation of the moveable handle slides the cutting tip on the moveable arm in contact with cutting surface on the stationary arm.

3. The rongeur of claim 2, wherein the stationary arm comprises a polymeric material.

4. The rongeur of claim 2, wherein the moveable arm comprises a polymeric material.

5. The rongeur of claim 2, wherein the moveable arm is selectively removable from the stationary arm.

6. The rongeur of claim 2, wherein the stationary handle, the stationary arm and the moveable arm comprises of polymeric materials.

7. The rongeur of claim 2, wherein the stationary arm consists of an injection molded polymeric material formed at least partially around a first metallic insert that forms the cutting tip and the moveable arm consists of an injection molded polymeric material formed at least partially around a second metallic insert that forms the cutting surface.

8. The rongeur of claim 2, wherein at least one of the cutting tip and cutting surface are modular.

9. The rongeur of claim 2, wherein the cutting tip of the moveable arm slides past the cutting surface of the stationary arm in a scissor-like motion in response to rotation of said moveable handle relative to said stationary handle.

10. The rongeur of claim 1, wherein the cutting tip of the moveable arm slides along a curved path relative to the cutting surface of the stationary arm in response to rotation of said moveable handle relative to said stationary handle.

11. The rongeur of claim 2, wherein the cutting tip of the moveable arm slides along an angled path relative to the cutting surface of the stationary arm in response to rotation of said moveable handle relative to said stationary handle, thereby creating a slicing motion between the cutting tip and the cutting surface.

12. The rongeur of claim 11, wherein at least a portion of the moveable arm proximate the cutting tip is spaced apart from a portion of the stationary arm, and the spaced apart portion of the moveable arm flexes towards the stationary arm when said moveable arm slides relative to said stationary arm.

13. The rongeur of claim 2, wherein the stationary arm flexes to reorient the cutting surface relative to the cutting tip.

14. The rongeur of claim 1 wherein the sliding movement between the moveable arm and the stationary arm in response to rotation of said moveable handle relative to said stationary handle is linear.

15. The rongeur of claim 1 wherein the sliding movement between the moveable arm and the stationary arm in response to rotation of said moveable handle relative to said stationary handle is non-linear.

16. A surgical rongeur comprising:
a moveable arm and a stationary body linked in a sliding relationship by a pair of spaced apart connecting guides;
the moveable arm having a cutting tip positioned integrally therein, the moveable arm comprising a polymeric material and the cutting tip comprising a metallic material;
the stationary body having a cutting surface positioned integrally therein, the stationary body comprising a polymeric material and the cutting surface comprising a metallic material;
the stationary body having an opening formed therethrough;
a moveable handle rotatably coupled to said stationary body, at least a portion of the moveable handle extending through the opening in the stationary body and connected to the moveable arm such that rotation of the moveable handle relative to the stationary body slides the moveable arm relative to the stationary body;
the stationary body further includes a first spring element formed integrally therein;
the moveable arm further includes a second spring element formed integrally therein; and
the first and second spring elements cooperating to bias the moveable arm to a first position relative to the stationary body in which the cutting tip is positioned at a first location that is separated from the cutting surface;
wherein rotation of the moveable handle relative to the stationary body slides the moveable arm relative to the stationary body so as to bring the cutting tip in close proximity to the cutting surface.

17. The rongeur of claim 16, wherein at least a portion of the moveable arm proximate the cutting tip is spaced apart from a portion of the stationary body proximate to the cutting surface, and at least one of the spaced apart connecting guides includes a ramped feature that flexes the portion of the moveable arm proximate the cutting tip towards the stationary body in response to rotation of the moveable handle relative to the stationary body.

18. The rongeur of claim 17, wherein the cutting tip of the moveable arm slides along a curved path relative to the cutting surface of the stationary body in response to rotation of said moveable handle relative to said stationary body.

19. The rongeur of claim 16, wherein the cutting tip of the moveable arm slides along an angled path relative to the cutting surface of the stationary body in response to rotation of said moveable handle relative to said stationary body, thereby creating a slicing motion between the cutting tip and the cutting surface.

20. The rongeur of claim 16, wherein the cutting tip of the moveable arm slides past the cutting surface of the stationary body in a scissor-like motion in response to rotation of said moveable handle relative to said stationary body.
